(19) United States
(12) Patent Application Publication
(10) Pub. No.: US 2026/0108378 A1
(43) Pub. Date: Apr. 23, 2026

(54) FEMALE EXTERNAL CATHETER DEVICES HAVING A URETHRAL CUP, AND RELATED SYSTEMS AND METHODS

(71) Applicant: PUREWICK CORPORATION, Covington, GA (US)

(72) Inventor: James David Hughett, SR., Conyers, GA (US)

(21) Appl. No.: 19/370,361

(22) Filed: Oct. 27, 2025

Related U.S. Application Data
(63) Continuation of application No. 17/996,064, now Pat. No. 12,472,090.
(60) Provisional application No. 63/011,657, filed on Apr. 17, 2020.

(51) Int. Cl. A61F 5/455; A61F 5/44

(52) U.S. Cl. CPC A61F 5/455; A61F 5/4401; A61F 5/4404

(57) ABSTRACT

Examples relate to fluid collection devices having a urethral cup, and related systems and methods. A fluid collection device includes a funnel, a body, a chamber outlet, and a fluid permeable structure. The funnel has a mouth with an outer periphery and narrows to a base defining an opening. The body includes a proximal end region and a distal end region distal to the funnel, and defines a chamber in fluid communication with the opening at the base of the funnel and extending from the opening beyond the outer periphery of the mouth of the funnel to the distal end region. The chamber outlet is in fluid communication with the chamber and positioned at the distal end region of the body. The fluid permeable structure is positioned within the chamber of the elongated body, and fills at least a portion of the chamber between the opening and the chamber outlet.

### CROSS-REFERENCE TO RELATED APPLICATIONS

This application is a continuation of U.S. application Ser. No. 17/996,064 filed Oct. 12, 2022, which is a U.S. Nationalization of PCT International Application No. PCT/US2021/027104 filed on 13 Apr. 2021, which claims priority to U.S. Provisional Patent Application No. 63/011,657 filed on Apr. 17, 2020, the disclosure of each of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters may be uncomfortable, painful, and may cause urinary tract infections.

Thus, users and manufacturers of fluid collection devices continue to seek new and improved devices, systems, and methods to collect urine.

### SUMMARY

Embodiments disclosed herein are related to fluid collection devices having a urethra cup, and related systems and methods. In an embodiment, a fluid collection device includes a funnel, a body, a chamber outlet, and a fluid permeable structure. The funnel has a mouth with an outer periphery and narrows to a base defining an opening. The body has a proximal end region and a distal end region distal to the funnel. The body defines a chamber in fluid communication with the opening at the base of the funnel and extends from the opening beyond the outer periphery of the mouth of the funnel to the distal end region. The chamber outlet is in fluid communication with the chamber and positioned at the distal end region of the body. The fluid permeable structure is positioned within the chamber of the elongated body and fills at least a portion of the chamber between the opening and the chamber outlet.

In an embodiment, a method of collecting urine includes positioning a funnel at a proximal end of a urine collection device between labia majora of the female user with the funnel covering a urethral opening of the female user. The method also includes collecting urine discharged in the funnel in a fluid permeable structure positioned in a chamber defined by a body of the urine collection device that is positioned between the labia majora and at least partially over the vagina of the female user. The chamber is in fluid communication with the funnel through an opening at a base of the funnel. The method also includes drawing urine from the chamber through a chamber outlet at a distal end region of the body with a conduit in fluid communication with the chamber and a vacuum source.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a block diagram of a fluid collection system, according to an embodiment.

FIG. 2A is an isometric view of a fluid collection device, according to an embodiment.

FIG. 2B is a cross-sectional view of the fluid collection device of FIG. 2A taken along line 2B-2B of FIG. 2A.

FIG. 2C is a rear view of the fluid collection device of FIG. 2A positioned over the urethra of a user.

FIG. 3 is a partial front isometric view of a fluid collection device, according to an embodiment.

FIG. 4A is an isometric view of a fluid collection device, according to an embodiment.

FIG. 4B is a cross-sectional view of the fluid collection device of FIG. 4A taken along line 4B-4B of FIG. 4A.

FIG. 4C is a side view of the fluid collection device of FIG. 4A in use on a user in a sitting position.

FIG. 4D is a side view of the fluid collection device of FIG. 4B in user on a user in a supine position.

FIG. 5 is a flow diagram of a method for collecting fluid, according to an embodiment.

### DETAILED DESCRIPTION

Embodiments disclosed herein are related to fluid collection devices having a urethral cup, and related systems and methods. Conventional female external catheter cups are typically configured for collection of urine via gravity, and may not effectively move urine in a vacuum system. Many of the female fluid collection devices according to one or more embodiments disclosed herein include a urethra cup (e.g., funnel) having one or more complex curves. This funnel with one or more complex curves allows the fluid collection device to better contact or be placed adjacent to at least one of the urethral or vaginal orifice(s). The fluid collection device may include a smaller size than conventional fluid collection devices because the funnel may be targeted towards a specific region of the genitalia with less dimensional variation. For example, the funnel may be positioned over the urethral opening of the user, and one or more portions of the labia of the user may hold or secure the funnel in place over the urethra. More specifically, the labia minora may close or be positioned around the funnel and the labia majoria may close or position around the body (or reservoir). The funnel may direct urine into chamber (e.g., a sump area) of the fluid collection device, which provides some overflow and also includes a permeable structure of material (e.g., a spun plastic) to keep the urine connected for transport via gravity and/or vacuum. This permeable structure may include a capillary functional element. In contrast to conventional devices, addition of the sump and spun plastic may aid in fluid removal in a vacuum mode.

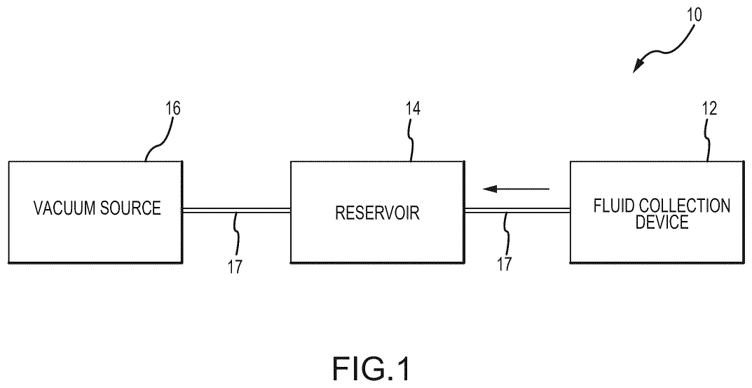

FIG. 1 is a block diagram of a fluid collection system 10, according to an embodiment. The fluid collection system 10 may be included in any of the embodiments of fluid collection systems described herein. The system 10 includes a fluid (e.g., urine) collection device 12 (e.g., any of the fluid collection assemblies disclosed herein), a urine collection container 14, and a pump 16 (or vacuum source). The fluid collection device 12, the urine collection container 14, and the pump 16 may be fluidly coupled to each other via one or more tubes 17 or conduits. For example, fluid collection device 12 may be operably coupled to one or more of the urine collection container 14 or the pump 16 via the tube 17. In some embodiments, the pump 16 may be secured directly to the urine collection container 14. Fluid (e.g., urine or other bodily fluids) collected in the fluid collection device 12 may be removed from the fluid collection device 12 via the tube 17 secured to the fluid collection device 12. Suction force may be introduced into the chamber of the fluid collection device 12 via the inlet of the tube 17 responsive to suction (e.g., vacuum) force applied at the outlet of the tube 17.

The suction force may be applied to the outlet of the tube 17 by the pump 16 either directly or indirectly. The suction force may be applied indirectly via the urine collection container 14. For example, the outlet of the tube 17 may be disposed within or fluidly coupled to an interior region of the urine collection container 14 and an additional tube 17 may extend from the urine collection container 14 to the pump 16. Accordingly, the pump 16 may apply suction to the fluid collection device 12 via the urine collection container 14. The suction force may be applied directly via the pump 16. For example, the outlet of the tube 17 may be disposed within the pump 16. An additional tube 17 may extend from the pump 16 to a point outside of the fluid collection device 12, such as to the urine collection container 14. In such examples, the pump 16 may be disposed between the fluid collection device 12 and the urine collection container 14.

The urine collection container 14 is sized and shaped to retain a fluid therein. The urine collection container 14 may include a bag (e.g., drainage bag), a bottle or cup (e.g., collection jar), or any other enclosed container for storing bodily fluid(s) such as urine. In some examples, the tube 17 may extend from the fluid collection device 12 and attach to the urine collection container 14 at a first point therein. An additional tube 17 may attach to the urine collection container 14 at a second point thereon and may extend and attach to the pump 16. Accordingly, a vacuum (e.g., suction) may be drawn through fluid collection device 12 via the urine collection container 14. Fluid, such as urine, may be drained from the fluid collection device 12 using the pump 16.

The pump 16 may include one or more of a manual vacuum pump, and electric vacuum pump, a diaphragm pump, a centrifugal pump, a displacement pump, a magnetically driven pump, a peristaltic pump, or any pump configured to produce a vacuum. The pump 16 may provide a vacuum or suction to remove fluid from the fluid collection device 12. In some examples, the pump 16 may be powered by one or more of a power cord (e.g., connected to a power socket), one or more batteries, or even manual power (e.g., a hand operated vacuum pump). In some examples, the pump 16 may be sized and shaped to fit outside of, on, or within the fluid collection device 12. For example, the pump 16 may include one or more miniaturized pumps or one or more micro pumps. The vacuum sources disclosed herein may include one or more of a switch, a button, a plug, a remote, or any other device suitable to activate the pump 16. Aspects of the fluid collection system 10 may be included in any of the fluid collection systems described herein. For example, the fluid collection device 12 may include any of the fluid or urine collection devices described herein.

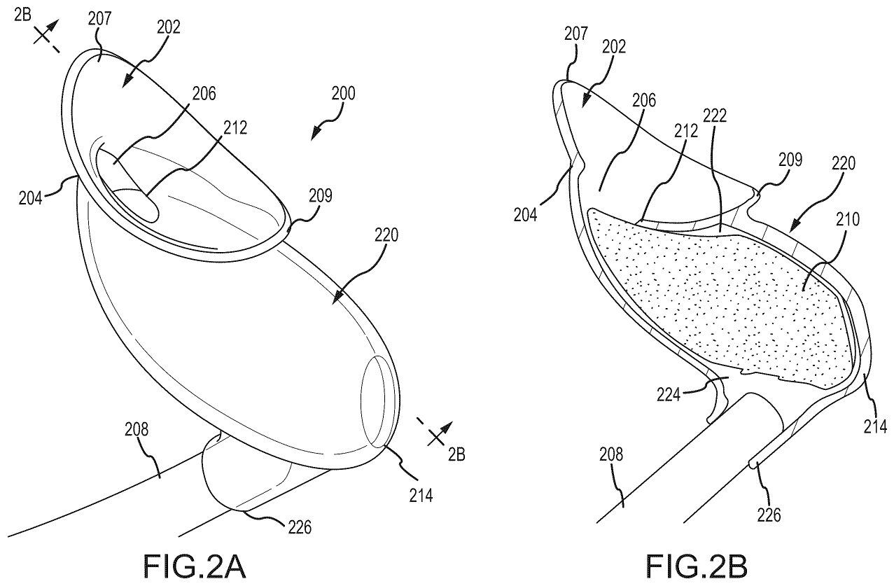

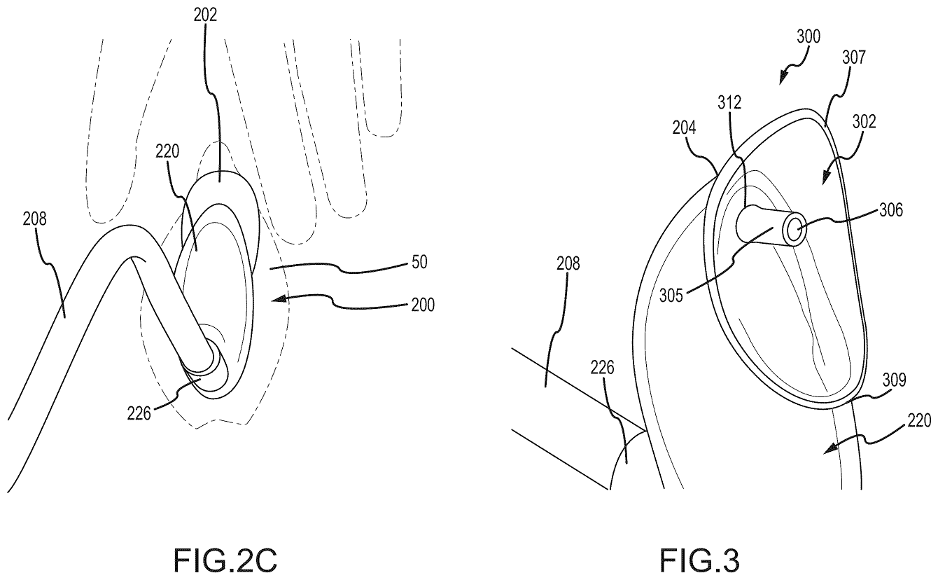

In many embodiments, the fluid collection device may be configured as a urine collection device. FIGS. 2A-2C, for example, are views of a female urine collection device 200, according to an embodiment. The urine collection device 200 includes a funnel 202, a body 220, and a fluid permeable structure 210. With specific reference to FIG. 2A, the funnel 202 may include a mouth 207 having or defining an outer periphery 209 of the funnel 202. The funnel 202 narrows from the mouth 207 to a base 212 that defines an opening 206. The funnel 202 may be sized to be positioned over the urethral opening of the user while one or more portions of the labia of the user hold or secure the funnel 202 in place over the urethra. More specifically, the labia minora may close and/or be positioned around the funnel 202 and the labia majora may close or position around the body 220.

In some embodiments, the mouth 207 defining the outer periphery 209 is substantially oval in shape. The mouth 207 may include a longitudinal length of about 1 cm to about 5 cm, about 1 cm to about 3 cm, about 3 cm to about 5 cm, about 1 cm to about 2 cm, about 2 cm to about 3 cm, about 3 cm to about 4 cm, about 4 cm to about 5 cm, less than about 5 cm, less than about 4 cm, less than about 3 cm, less than about 2 cm, or less than about 1 cm. The mouth 207 may include a lateral width of about 0.5 cm to about 4 cm, about 0.5 cm to about 2 cm, about 2 cm to about 4 cm, about 0.5 cm to about 1 cm, about 1 cm to about 1.5 cm, about 1.5 cm to about 2 cm, about 2.5 cm to about 3 cm, about 3 cm to about 3.5 cm, about 3.5 cm to about 4 cm, less than about 4 cm, less than about 3.5 cm, less than about 3 cm, less than about 2.5 cm, less than about 2 cm, less than about 1.5 cm, less than about 1 cm, or less than about 0.5 cm. The funnel 202 may include a depth from the mouth 207 to the opening 206 of about 0.25 cm to about 3 cm, about 0.25 cm to about 1.25 cm, about 1 cm to about 2 cm, about 2 cm to about 3 cm, about 0.25 cm to about 0.5 cm, about 0.5 cm to about 0.75 cm, about 0.75 cm to about 1 cm, about 1 cm to about 1.25 cm, about 1.25 cm to about 1.5 cm, about 1.5 cm to about 1.75 cm, about 1.75 cm to about 2 cm, about 2 cm to about 2.25 cm, about 2.25 cm to about 2.5 cm, about 2.5 cm to about 2.75 cm, or about 2.75 cm to about 3 cm.

The urine collection device 200 also includes a body 220 having a proximal end region 204 and a distal end region 214 distal to the funnel 202. Turning to FIG. 2B, the body 202 may define a chamber 222 in fluid communication with the opening 206 at the base 212 of the funnel 202 and extending from the opening 206 at least beyond the outer periphery 209 of the mouth 207 of the funnel 202 to the distal end region 214. The funnel 202 may direct urine discharged into the funnel 202 into chamber 222 (e.g., a sump area) through the opening 206. The chamber 222 may provide some overflow capacity and also may include a fluid permeable structure 210 of material (e.g., a spun plastic) to keep the urine connected for transport via gravity and/or vacuum. This fluid permeable structure 210 may include a capillary functional element. In contrast to conventional devices, addition of the chamber 222 and fluid permeable structure 210 may aid in fluid removal in a vacuum mode.

The body 220 of the urine collection device 200 may be substantially ovoid-or egg-shaped, or another suitable shape. The body 220 may include a longitudinal length of about 1 cm to about 7 cm, about 1 cm to about 3 cm, about 3 cm to about 5 cm, about 5 cm to about 7 cm, about 1 cm to about 2 cm, about 2 cm to about 3 cm, about 3 cm to about 4 cm, about 4 cm to about 5 cm, about 5 cm to about 6 cm, about 6 cm to about 7 cm, less than about 7 cm, less than about 6 cm, less than about 5 cm, less than about 4 cm, less than about 3 cm, less than about 2 cm, or less than about 1 cm. The body 220 may include a lateral width or diameter of about 0.5 cm to about 4 cm, about 0.5 cm to about 2 cm, about 2 cm to about 4 cm, about 0.5 cm to about 1 cm, about 1 cm to about 1.5 cm, about 1.5 cm to about 2 cm, about 2.5 cm to about 3 cm, about 3 cm to about 3.5 cm, about 3.5 cm to about 4 cm, less than about 4 cm, less than about 3.5 cm, less than about 3 cm, less than about 2.5 cm, less than about 2 cm, less than about 1.5 cm, less than about 1 cm, or less than about 0.5 cm.

The urine collection device 200 also may include a chamber outlet 224 in fluid communication with the chamber 222 and positioned at the distal end region 214 of the body 220. The urine collection device 200 may include a conduit port 226 at the distal end region 214 extending from the chamber outlet 224 and configured to secure the urine collection device 200 to a conduit 208 or tube. The chamber outlet 224 may be defined at least partially by the distal end region 214 and/or the conduit port 226.

The fluid permeable structure 210 may be positioned within the chamber 222 of the body 220. In some embodiments, the fluid permeable structure 210 substantially fills the chamber 222 and/or is shaped complementary to the chamber 222 and the body 220. For example, the fluid permeable structure 210 may be substantially ovoid-or egg-shaped substantially complementary to the chamber 222 and the body 220. In some embodiments, the fluid permeable structure 210 is positioned adjacent and/or interfacing the opening 206 such that a portion of the fluid permeable structure 210 extends across at least a portion of the opening 206. The fluid permeable structure 210 may be spaced from the conduit port 226 and/or the conduit 208 secured to the conduit port 226.

The fluid permeable structure 210 can be configured to wick and/or allow transport of fluid away from the opening 206, thereby preventing the fluid from escaping the funnel 202. The fluid permeable structure 210 also can wick and/or allow transport of the fluid generally towards the chamber outlet 224. The fluid permeable structure 210 can include any material that can wick and/or allow transport of the fluid. The permeable properties referred to herein can be wicking, capillary action, diffusion, or other similar properties or processes, and are referred to herein as “permeable” and/or “wicking.” Such “wicking” or other physical properties may exclude absorption into the fluid permeable structure 210, such as not include adsorption of the bodily fluids into the fluid permeable structure 210. Put another way, substantially no absorption or solubility of the bodily fluids into the material may take place after the material is exposed to the bodily fluids and removed from the bodily fluids for a time. While no absorption or solubility is desired, the term “substantially no absorption” may allow for nominal amounts of absorption and/or solubility of the bodily fluids into the fluid permeable structure 210 (e.g., absorbency), such as less than about 30 wt % of the dry weight of the fluid permeable structure 210, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the fluid permeable structure 210. In an embodiment, the fluid permeable structure 210 may include at least one absorbent or adsorbent material.

The fluid permeable structure 210 can include a one-way fluid movement fabric. As such, the fluid permeable structure 210 can remove fluid from the area around the urethra, thereby leaving the area and urethra dry. The fluid permeable structure 210 can enable the fluid to flow generally towards the chamber outlet 224 and the conduit 208 in fluid communication with the chamber 204. The fluid permeable structure 210 can include a porous or fibrous material, such as hydrophilic polyolefin. In some embodiments, the fluid permeable structure 210 consists of or consists essentially of a porous or fibrous material, such as hydrophilic polyolefin. Examples of polyolefin that can be used in the fluid permeable structure 210 include, but are not limited to, polyethylene, polypropylene, polyisobutylene, ethylene propylene rubber, ethylene propylene diene monomer, or combinations thereof. Moreover, the fluid permeable structure 210 can be manufactured according to various manufacturing methods, such as molding, extrusion, or sintering. The fluid permeable structure 210 can include varying densities or dimensions.

In some embodiments, the fluid permeable structure 210 can include two or more layers of fluid permeable materials. For example, the fluid permeable structure 210 can include a fluid permeable membrane covering or wrapped around a fluid permeable support, with both the fluid permeable membrane and the fluid permeable support being disposed in the chamber 204. The fluid permeable membrane can cover or extend across at least a portion (e.g., all) of at least the side of the fluid permeable support facing the penis of the user. The fluid permeable membrane and the fluid permeable support can be configured to wick any fluid away from the opening 206, thereby preventing the fluid from escaping the opening 206 and promoting removal of the fluid through the conduit 208.

The fluid permeable membrane and the fluid permeable support also can wick and/or allow transport of the fluid generally towards an interior of the chamber 204, such as the chamber outlet 224. The fluid permeable membrane can include any material that can wick the fluid. For example, the fluid permeable membrane can include fabric, such as a gauze (e.g., a silk, linen, polymer based materials such as polyester, or cotton gauze), nylon (such as a spun nylon fibers), another soft fabric (e.g., jersey knit fabric or the like), or another smooth fabric (e.g., rayon, satin, or the like). Forming the fluid permeable membrane from gauze, soft fabric, and/or smooth fabric can reduce chaffing caused by the urine collection device 200. Other embodiments of fluid permeable membranes and fluid permeable supports are disclosed in U.S. patent application Ser. No. 15/612,325 filed on Jun. 2, 2017; U.S. patent application Ser. No. 15/260,103 filed on Sep. 8, 2016; U.S. patent application Ser. No. 15/611,587 filed on Jun. 1, 2017; PCT Patent Application No. PCT/US19/29608, filed on Apr. 29, 2019, the disclosure of each of which is incorporated herein, in its entirety, by this reference. In many embodiments, the fluid permeable structure 210 includes a fluid permeable support including a porous spun nylon fiber structure and a fluid permeable wicking membrane including gauze at least partially enclosing the spun nylon fiber structure. For example, the fluid permeable structure 210 may include a gauze or other wicking fabric positioned to contact the skin of the user through the opening 206. In some embodiments, the gauze or other wicking fabric is wrapped around a body of spun nylon fibers material. In some embodiments, the gauze is absent, and the fluid permeable structure 210 includes the spun nylon fibers material.

The urine collection device 200 also includes a conduit 208 (e.g., tube) removably secured or securable to the conduit port 226 effective to allow fluid communication between the conduit 208 and the chamber 204 through the chamber outlet 224. The conduit 208 may be configured to provide fluid communication between a vacuum source and the chamber 222. The conduit 208 may extend at least partially through the conduit outlet 224 and into the chamber 222. In some embodiments, the conduit 208 may contact the fluid permeable structure 210 such that at least a portion of the fluid permeable structure 210 covers at least a portion (e.g. all) of an opening or inlet in the conduit 208. Fluid discharged into the funnel 202 and collected in the fluid permeable structure 210 may flow to and pool in the sump region or chamber outlet 224 for removal when the vacuum is applied on the conduit 208.

Each of the funnel 202, the body 220, and the conduit outlet 226 may include a fluid impermeable barrier. The fluid impermeable barrier may include a substantially flexible fluid impermeable material, such as a fluid impermeable polymer (e.g., silicone, polypropylene, polyethylene, polyethylene terephthalate, a polycarbonate, etc.), polyurethane films, thermoplastic elastomer, oil, another suitable material, or combinations thereof. In some embodiments, the fluid impermeable barrier includes a paper-like fluid impermeable material or a fluid impermeable fabric. In some embodiments, the fluid impermeable barrier is substantially rigid.

FIG. 2C is a rear view of the urine collection device 200 positioned over the urethral opening of a female user. The mouth 207 of the funnel 202 may be sized to fit between at least the labia majora of a female user and cover a urethral opening of the female user. In some embodiments, mouth 207 of the funnel 202 is sized to fit between the labia minora 50 of the female user and cover the urethral opening of the female user. The body 220 may cover at least a portion (e.g., all) of the vaginal opening. The labia minora 50 and/or the labia majora may assist in retaining the funnel 202 in place over the urethral opening such that urine discharged by the user enters the funnel 202 and is directed to the opening 206 at the base 212 of the funnel 202. Urine may then enter and/or be drawn into the chamber 222 and the chamber outlet 224 for removal through the conduit 208.

FIG. 3 is a partial front isometric view of a urine collection device 300, according to an embodiment. Unless otherwise noted, the urine collection device 300 may include any aspect of the urine collection device 200, such as the body 220 defining the chamber 222, the conduit port 226, the chamber outlet 224 (not visible), and the fluid permeable structure 210 (not visible). The urine collection device 300 also includes a funnel 302. Unless otherwise noted, the funnel 302 may include any aspect of the funnel 202. For example, the funnel 302 may include a base 312, an opening (not visible) at the base 312, and a mouth 307 distal to the base 312 and defining an outer periphery 309.

The funnel 302 also may include a tip 305 extending from the base 312 of the funnel 302 towards the mouth 307. The tip 305 defines a channel 306 extending through the tip 305 and providing fluid communication between the funnel 302 and the opening leading to the chamber of the body 220. The tip 305 may be sized to fit at least partially within the urethral opening of the female user such that urine being discharged by the user enters directly into the channel 306 of the tip 305 and flows into the chamber of the body 220.

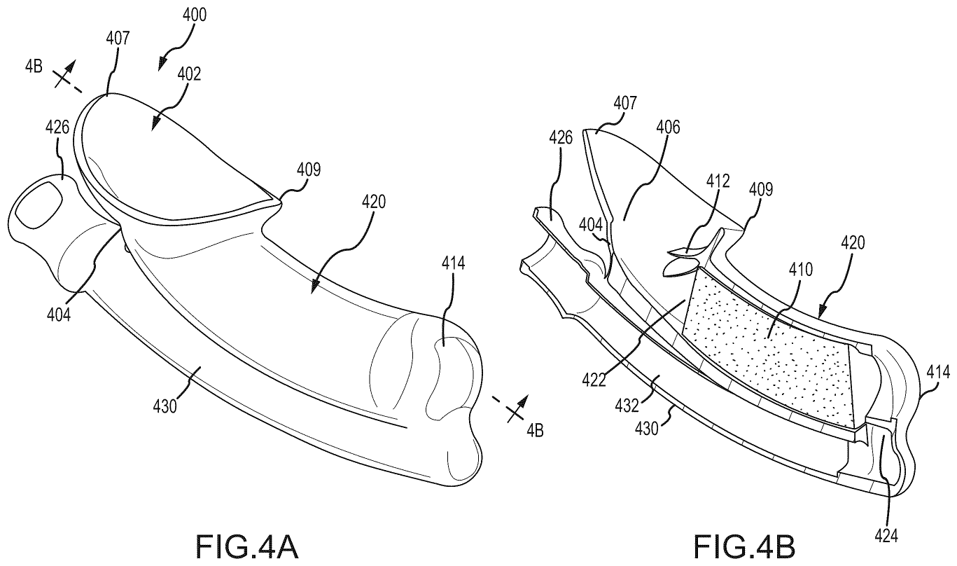

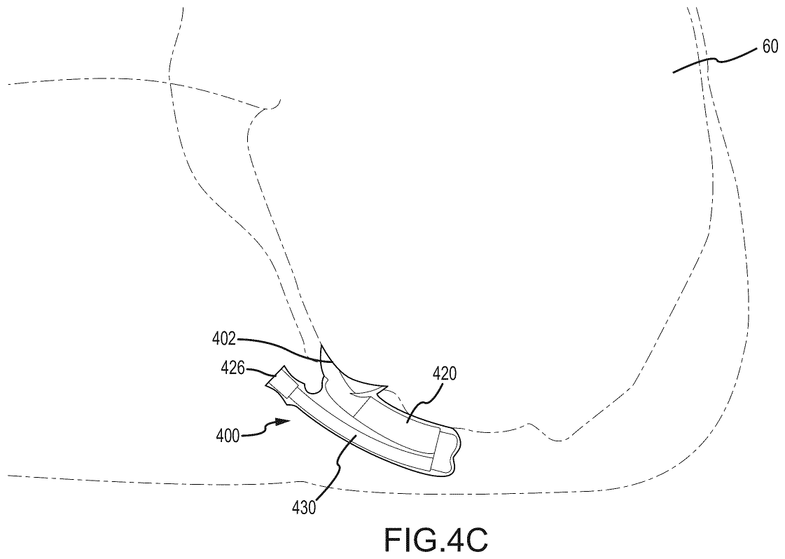

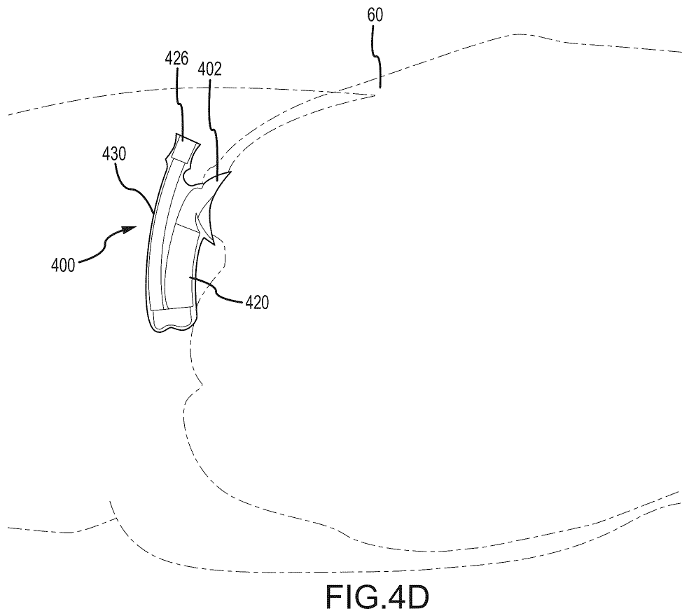

FIGS. 4A and 4B are views of an embodiment of a urine collection device 400. Unless otherwise noted, the urine collection device 400 may include any aspect of the urine collection devices 200, 300. For example, the urine collection device 400 may include a funnel 402 having a mouth 407 with an outer periphery 409 and narrowing to a base 412 defining an opening 406. The urine collection device 400 also may include a body 420 having a proximal end region 404 and a distal end region 414 distal to the funnel 402. The body 420 may define a chamber 422 in fluid communication with the opening 406 at the base 412 of the funnel 402. The body 420 also may extend from the opening 406 beyond the outer periphery 409 of the mouth 407 of the funnel 402 to the distal end region 414 of the body 420. In some embodiments, the body 420 is substantially cylindrical. The urine collection device 400 also may include a chamber outlet 424 in fluid communication with the chamber 422 and positioned at the distal end region 414 of the body 420. The urine collection device 400 also may include a fluid permeable structure 410 positioned within the chamber 422 of the body 420. The fluid permeable structure 420 may fill at least a portion of the chamber 422 between the opening 406 and the chamber outlet 424. Though not shown, the urine collection device 400 also may include a tip 305.

The urine collection device 400 also may include a tubular member 430 extending from the distal end region 414 towards the proximal end region 404 with the body 420 being positioned between the funnel 402 and the tubular member 430. The tubular member 430 may define a passage 432 in fluid communication with the chamber 422 through the chamber outlet 424. In some embodiments, the tubular member 430 may extend from the distal end region 414 at least the longitudinal length of the body 420 to the proximal end region 404 of the body 420. Both the body 420 and the tubular member 430 may be arched substantially complementary to one another. In some embodiments, the body 420 may include a diameter that is greater than a diameter of the tubular member 430.

The urine collection device 400 may include a conduit port 426 on the tubular member 430 distal to the chamber outlet 424. The conduit port 426 is configured to secure the urine collection device 400 to the conduit 208 (not shown). When secured to the conduit port 426, the tube 208 may provide fluid communication between a vacuum source and the chamber 422 through the chamber outlet 424 and the passage 432 of the tubular member 430.

The urine collection device 400 may be used similar to the urine collection device 300. For example, the mouth 407 of the funnel 402 may be sized to fit between at least the labia majora of a female user and cover a urethral opening of the female user. In some embodiments, mouth 407 of the funnel 402 is sized to fit between the labia minora of the female user and cover the urethral opening of the female user. The body 420 may cover at least a portion (e.g., all) of the vaginal opening. The labia minora and/or the labia majora may assist in retaining the funnel 402 in place over the urethral opening such that urine discharged by the user enters the funnel 402 and is directed to the opening 406 at the base 412 of the funnel 402. Urine may then enter and/or be drawn into the chamber 422 and the chamber outlet 424 for removal through passage 432 and the conduit 208.

The urine collection device 400 (and the urine collection devices 200, 300) may be used by the user in multiple body positions. FIG. 4C, for example, shows the urine collection device 400 with the funnel 402 positioned over or otherwise covering the urethral opening of a user 60 with the user in a sitting position. FIG. 4D shows the urine collection device 400 with the funnel 402 positioned over or otherwise covering the urethral opening of the user with the user in a supine position. The tubular member 430 may direct the conduit 208 away from the body of the user 60, and inhibit kinking or pinching of the conduit 208. In either the sitting or supine positions, the anatomy of the user (e.g., the labia minor and/or the labia majora) may at least assist in retaining the funnel 402 in a position to cover the urethral opening of the user 60.

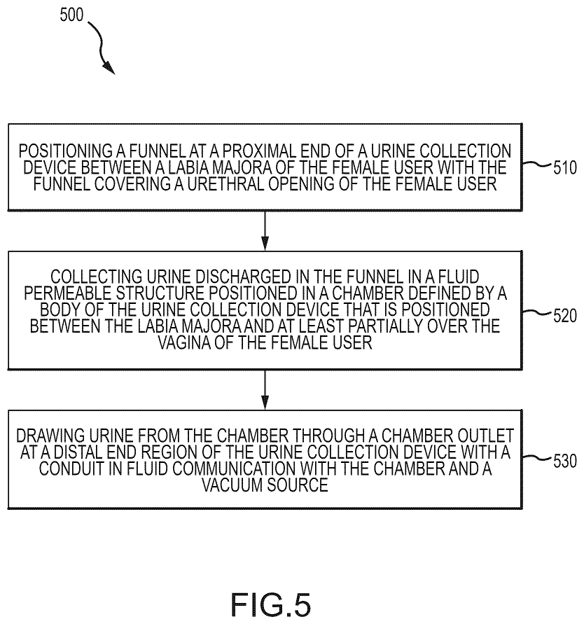

FIG. 5 is a flow diagram of a method 500 for collecting fluid from a user, according to an embodiment. The method 500 includes an act 510 of positioning a funnel at a proximal end of a urine collection device between labia majora of the female user with the funnel covering a urethral opening of the female user. The method 500 also includes an act 520 of collecting urine discharged in the funnel in a fluid permeable structure positioned in a chamber defined by a body of the urine collection device that is positioned between the labia majora and at least partially over the vagina of the female user. The chamber may be in fluid communication with the funnel through an opening at a base of the funnel. The method 500 also includes an act 530 of drawing urine from the chamber through a chamber outlet at a distal end region of the body with the conduit in fluid communication with the chamber and a vacuum source.

The act 530 of drawing urine from the chamber through a chamber outlet at a distal end region of the body with a conduit in fluid communication with the chamber and a vacuum source may include drawing the urine from the chamber through the chamber outlet at the distal end region of the body with the conduit in fluid communication with the chamber and the vacuum source when the female user is in a sitting position. The act 530 of drawing urine from the chamber through a chamber outlet at a distal end region of the body with a conduit in fluid communication with the chamber and a vacuum source may include drawing the urine from the chamber through the chamber outlet at the distal end region of the body with the conduit in fluid communication with the chamber and the vacuum source when the female user is in a supine position.

In some embodiments, the method 500 also may include securing the conduit to a conduit port on the tubular member. In some embodiments, the method 500 also may include inserting a tip in the funnel into the urethral opening, the tip defining a channel in fluid communication with the opening.

The acts of the method 500 described above are for illustrative purposes. For example, the acts of the method 500 can be performed in different orders, split into multiple acts, modified, supplemented, or combined. In an embodiment, one or more of the act of the method 500 can be omitted from the method 500. Any of the acts of the method 500 can include using any of the portable urine collection systems disclosed herein.

As used herein, the term “about” or “substantially” refers to an allowable variance of the term modified by “about” or “substantially” by ±10% or +5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting.

1. A urine collection device, comprising:
a body having a proximal end region at least partially defining an opening and a distal end region, the body defining a chamber in fluid communication with the opening and extending from the opening to the distal end region;
a chamber outlet in fluid communication with the chamber and positioned at the distal end region of the body;
and
a conduit port configured to secure the urine collection device to a conduit and positioned proximal of the chamber outlet such the conduit secured to the conduit port is oriented at least partially proximal from the distal end region of the body,
wherein the body is sized and dimensioned to fit between labia majora of a female user effective to allow the labia majora to close or position at least partially around the body.

2. The urine collection device of claim 1, further comprising the conduit in fluid communication with the chamber outlet and configured to provide fluid communication between a vacuum source and the chamber.

3. The urine collection device of claim 1, wherein the conduit port extends at least partially proximal from the chamber outlet.

4. The urine collection device of claim 1, further comprising a tubular member secured to a back side of the body opposite of the opening and extending from the distal end region towards the proximal end region, the tubular member defining a passage in fluid communication with the chamber through the chamber outlet and the conduit being positioned on the tubular member proximal of the chamber outlet, wherein the tubular member and the body are sized and dimensioned to fit between the labia majora of the female user.

5. The urine collection device of claim 4, wherein the body and the tubular member are arched complementary to one another.

6. The urine collection device of claim 4, wherein the body is substantially cylindrical and includes a diameter greater than a diameter of the tubular member.

7. The urine collection device of claim 4, wherein the tubular member extends from the distal end region at least to the proximal end region of the body.

8. The urine collection device of claim 7, wherein the conduit port on the tubular member is disposed proximal of the opening.

9. The urine collection device of claim 1, further comprising a funnel having a mouth with an outer periphery and narrowing to a base at least partially defining the opening, wherein the mouth of the funnel is sized to fit between the labia majora of the female user and cover a urethral opening of the female user.

10. The urine collection device of claim 9, further comprising a tip extending from the base of the funnel towards the mouth, the tip being sized to fit at least partially within the urethral opening of the female user and defining a channel in fluid communication with the opening.

11. The urine collection device of claim 1, further comprising a fluid permeable structure positioned within the chamber of the elongated body, the fluid permeable structure filling at least a portion of the chamber between the opening and the chamber outlet.

12. A urine collection device, comprising:
a body having a proximal end region at least partially defining an opening on a front side of the body and a distal end region, the body defining a chamber in fluid communication with the opening and extending from the opening to the distal end region;
a tubular member secured to a back side of the body opposite of the front side and extending from the distal end region towards the proximal end region, the tubular member defining a passage in fluid communication with the chamber; and
a conduit port positioned on the tubular member proximal of the distal end region of the body and configured to secure the urine collection device to a conduit with the conduit oriented at least partially proximal from the distal end region of the body,
wherein the tubular member and the body are sized and dimensioned to fit between the labia majora of the female user effective to allow the labia majora to close or position at least partially around the body.

13. The urine collection device of claim 12, further comprising the conduit in fluid communication with the chamber of the body and the passage of the tubular member, the conduit configured to provide fluid communication between a vacuum source and the chamber.

14. The urine collection device of claim 12, wherein the body and the tubular member are arched complementary to one another.

15. The urine collection device of claim 12, wherein the body is substantially cylindrical and includes a diameter greater than a diameter of the tubular member.

16. The urine collection device of claim 12, wherein the tubular member extends from the distal end region at least to the proximal end region of the body.

17. The urine collection device of claim 16, wherein the conduit port on the tubular member is disposed proximal of the proximal end region of the body.

18. The urine collection device of claim 12, further comprising a funnel having a mouth with an outer periphery and narrowing to a base at least partially defining the opening, wherein the mouth of the funnel is sized to fit between the labia majora of the female user and cover a urethral opening of the female user.

19. The urine collection device of claim 18, further comprising a tip extending from the base of the funnel towards the mouth, the tip being sized to fit at least partially within the urethral opening of the female user and defining a channel in fluid communication with the opening.

20. The urine collection device of claim 12, further comprising a fluid permeable structure positioned within the chamber of the elongated body, the fluid permeable structure filling at least a portion of the chamber between the opening and the chamber outlet.
